(19) United States
(12) Patent Application Publication
(10) Pub. No.: US 2023/0364204 A1
(43) Pub. Date: Nov. 16, 2023

(54) Bioactive Peptides, Compositions, Production Process, and Use of Bioactive Peptides as Anti-Tumoral Agents

(71) Applicant: PHP BIOTECH INTERNATIONAL INC., Keller, TX (US)

(72) Inventors: Moacyr Ramos BIGHETTI, São Paulo (BR); Patricia Heloise Alves BEZERRA, São Paulo (BR)

(73) Assignee: PHP BIOTECH INTERNATIONAL INC., Keller, TX (US)

(21) Appl. No.: 18/313,141

(22) Filed: May 5, 2023

Related U.S. Application Data
(60) Provisional application No. 63/341,339, filed on May 12, 2022.

(51) Int. Cl. A61K 38/46; A61P 35/04; C12N 9/18

(52) U.S. Cl. CPC A61K 38/465; A61P 35/04; C12N 9/18; C12Y 301/01004

(57) ABSTRACT

The present disclosure relates to bioactive peptides, compositions, production processes and uses of bioactive peptides based on snake venom as anti-tumoral agents. The disclosed bioactive peptides are synthesized based on the C-terminal of the Crotoxin B and its derivatives. The disclosed peptides demonstrate a lessened toxic effect and an elevated anti-tumoral activity against tumor cells, but specifically against triple-negative breast cancer cells, as compared to current standard care. Therefore, the disclosed bioactive snake venom peptides derived from Crotoxin are for the modulation of cellular proliferation, apoptosis, necrosis and the cycle progression of the tumor cells, especially in the triple negative breast cancer cells, presenting a suitable alternative for the sole or complementary treatment of patients with aggressive solid tumors.

### CROSS-REFERENCE TO RELATED APPLICATION

This application claims priority to U.S. Provisional Pat. Application No. 63/341,339 filed May 12, 2022, the contents of which are incorporated herein in their entirety.

### STATEMENT PURSUANT TO 37 CFR 1.77(B)(5)

Accompanying this application is an .XML file containing SEQ IDs 1-10 as are recited at the end of this application and in the claims which material is incorporated by reference herein as if fully set forth in this application.

### FIELD OF THE INVENTION

The present invention provides bioactive peptides, compositions, production process and use of bioactive peptides based on snake venom as anti-tumoral agents.

### BACKGROUND OF THE INVENTION

Data from the American Cancer Society supports the expectation that 1.9 million new cases of cancer and 609,360 deaths from cancer will occur in 2022 in the U.S.

Irrespective of the fact that the risk of dying of cancer has dropped in the last few years, there continues to exist highly aggressive and life-ending types of cancer that could benefit from new alternative forms of treatment.

One of these life-ending cancers is the triple negative breast cancer, which accounts for 10-15% of all breast cancers. This type of cancer is different from the other known breast cancers, as it grows and spreads faster, has a worse prognosis for survival and suffers from the limited availability of treatment options. Indeed the nomenclature used for this cancer type (triple negative) makes reference to the negative results in the three tests applied to detect the presence of estrogen or progesterone receptors or HER2 protein in the breast cancer cells.

Treatments available for triple negative breast cancer include chemotherapy with usual chemo drugs such as anthracyclines, (e.g. doxorubicin) taxane, capecitabine, gemcitabine, eribulin, among others. Surgery and radiation are also treatment options, depending on certain tumor features.

However, due to severe side effects, toxicities and low specificity, new alternative options for the treatment of aggressive solid breast cancer tumors in patients are highly desirable.

Certain diseases, including specific types of cancer, could be treated using specific proteins and peptides (protein fragments) having biological activity in humans. It is well-known that those bioactive proteins and peptides can have their origins in other animals, such as terrestrial mammals, marine animals, amphibians, and animal venoms.

The first toxic animal venom component to be purified and crystallized was Crotoxin (i.e. Crotalus toxin or CTX), the major venom component from Crotalus durissus terrificus, also known as South American rattlesnake.

This component is composed of a heterodimeric complex of two proteins, the first being a basic, toxic phospholipase A2 (PLA₂) also known as Component B or CB and, the second one, an acidic, non-toxic, non-enzymatic component (crotapotin) also known as Component A or CA, which acts as a chaperon protein in a synergistic way, potentiating the toxicity of PLA₂. There are different isoforms of crotoxin and individual variations in the venom levels of this protein. The expression of different isoforms in the same venom gland may serve to maximize the snake venom protein activity. It was shown that the CA and CB isoforms can form at least 16 different CTX complexes. Also, it has been shown that different isoforms of CB show differences in the enzymatic and pharmacological activities of the CTX. Thus, CTX isoforms were classified into two classes (I and II) depending on their toxicity and enzymatic activity. The CB b, CB c and CB d isoforms complexed to CA are more toxic, have less enzymatic activity and dissociate from CA (class I isoforms) more slowly compared to CB a2 isoform (class II isoforms).

It is known from the state of art that the crotoxin protein contains different molecular regions responsible for specific pharmacological activities - neurotoxicity, myocity, nephrotoxicity and cardiotoxicity are the most common activities of this protein in vivo. Specifically, CB binds to receptors at the presynaptic membrane of several organisms, inhibiting the release of acetylcholine and, consequently, promoting neuromuscular blockade. However, it is important to highlight that, until recently, the exact location of the active sites in the structure of the snake venom responsible for its toxic activity remained unclear, being attributed to the N-terminal, IBS (interfacial binding surface) or C-terminal in different independent international studies.

The present invention is the first one to relate the synthetic C-terminal of the Crotoxin B and its derivatives with a lessened toxic effect and an elevated anti-tumoral activity against tumor cells but specifically against triple-negative breast cancer cells.

Our studies showed that other peptides, such as those located in the N-terminal region of Crotoxin B, did not show a promising effect.

Moreover, the in vitro and in vivo assays showed that the identified peptide can affect the cellular proliferation, apoptosis, necrosis and the cycle progression of the tumor cells, especially in the triple negative breast cancer cells.

Therefore, the present invention describes a bioactive snake venom peptide derived from crotoxin for the modulation of cellular proliferation, apoptosis, necrosis and the cycle progression of the tumor cells, especially in the triple negative breast cancer cells, presenting a suitable alternative for the sole or complementary treatment of patients with aggressive solid tumors.

### SUMMARY

The present invention describes a non-toxic bioactive snake venom peptide used for the treatment of aggressive solid tumors.

In another aspect, the invention comprises a bioactive peptide derived from the C-terminal of CB protein of Crotalus durissus terrificus.

In one aspect, the invention comprises a bioactive peptide in a substantially pure form (>95%).

More specifically, the bioactive peptide is selected from the group comprising the SEQ ID 1, SEQ ID 2, SEQ ID 3, SEQ ID 4, SEQ ID 5, SEQ ID 6, SEQ ID 7,SEQ ID 8, SEQ ID 9 and/or SEQ ID 10.

The bioactive peptide could be also selected from the group comprising at least 70% of similarity with the bioactive peptides SEQ ID 1, SEQ ID 2, SEQ ID 3, SEQ ID 4, SEQ ID 5, SEQ ID 6, SEQ ID 7,SEQ ID 8, SEQ ID 9 and/or SEQ ID 10.

Additionally, the bioactive peptide can be inserted in a pharmaceutical composition.

In one aspect, the isolated bioactive peptide or the composition comprising the bioactive peptide is used for the modulation of cellular proliferation, apoptosis, necrosis and/or the cycle progression in tumor cells.

Specifically, the peptide is therapeutically effective for the treatment of aggressive solid tumors, such as lung, prostate, eye cancer (ocular melanoma), colorectal, skin, brain, pancreas, kidney or breast cancer. More specifically, it is used for the treatment of triple negative breast cancer patients.

In a different aspect, the invention claims a production process of the a bioactive peptide selected from the group comprising SEQ ID 1, SEQ ID 2, SEQ ID 3, SEQ ID 4, SEQ ID 5, SEQ ID 6, SEQ ID 7,SEQ ID 8, SEQ ID 9 and/or SEQ ID 10 is synthetically obtained, comprising the following steps:
a) amino acids coupling and high efficiency synthesis assisted by microwaves;
b) purification of the peptide was performed using HPLC (High efficiency liquid chromatography);
c) analysis of the results by mass spectroscopy.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The disclosure will be more clearly understood by reference to the Figures, as listed:

FIG. 1 shows the HPLC characterization of 3-NantC, where a purity higher than 95% (95.15%) was observed for this batch.

FIG. 2 shows the MS/MS characterization that confirmed the 3-NAntC peptide (SEQ ID 1) sequence through the m/z relation.

FIG. 3 illustrates the untreated triple negative tumor cells, presented in large numbers.

FIG. 4 illustrates the tumor cells after being treated with the peptide 3-NantC (SEQ ID 1) for 24h. The arrows indicate the tumor cells, which are shown in smaller numbers and with vesicles when compared with the untreated tumor cells.

FIG. 5 represents the result of the MTT assay for both MDAMB231 cells (triple negative) and MCF10A cells (mammary benign cells) treated with the peptides (Peptides 1 and 2) originated from the N-terminal regions of the Crotoxin B, wherein no antitumor effect or high toxicity was noted in the tumor cells.

FIG. 6 illustrates the result of the MTT Assay, where decreased viability was indicated for the tumor cell line, while the fibroblast (benign) cell line remained alive in the tests for 24, 48 and 72 hours.

FIG. 7 represents the comparison of the antitumor effect of the 3-NAntC to commercial drugs (doxorubicin and cisplatin) through the results of the MTT assay.

FIG. 8 presents the results of the BrdU labeling, which shows a decreased proliferation of triple negative tumor cells when treated with 3-NAntC, even at concentrations that did not show cytotoxicity (e.g., 0.2 µg/mL).

FIG. 9 shows the cell cycle progression assay at 48h, in which 3-NAntC treatment causes a decrease in G0/G1 phase while it provokes a G2/M arrest.

FIG. 10 presents the lactate dehydrogenase release of MDAMB231 cells after treatment with the 3-NAntC peptide for 24, 48 and 72h. There is no significant difference in this biomarker, suggesting no involvement of either necrosis, necroptosis or pyroptosis as a primary cell death mechanism.

FIG. 11 shows a comparison of the MTT assay of MDAMB231 cells treated with 3-NAntC only or altogether with the autophagy inhibitor 3-methyladenine (3-MA). Since no difference was observed, the 3-NAntC peptide does not primarily induce cell death by autophagy mechanism.

FIG. 12 presents the results of the flow cytometry labeled with Annexin V-FITC and Propidium Iodide (PI), which indicates that 3-NAntC induces cell death by apoptosis with a very low amount of necrosis.

FIG. 13 presents the cell viability of MDAMB231 cells in front of various 3-NAntC forms (SEQ2-10), indicating that linear forms have interesting antitumor effects as well as the monomeric form.

FIG. 14 presents the cell viability of MDAMB231 cells when SEQ IDs 4 and 5 are combined. Even though there is a decrease in cell viability, 3-NAntC has a better antitumor effect.

FIG. 15 shows the great tolerability of the 3-NAntC peptide in Zebrafish.

FIG. 16 shows the antitumor and the antimetastatic effects of high doses of 3-NAntC against the xenograft model of MDAMB231 in Zebrafish.

FIG. 17 shows that even at low doses of 3-NAntC the in vivo antimetastatic effect of 3-NAntC remains.

### DETAILED DESCRIPTION OF THE INVENTION

The term “bioactive peptide(s)” comprises the C-terminal part of the crotoxin protein obtained from Crotalus durissus terrificus and its derivatives. In a specific way, the bioactive peptide(s) shows high purity (more than 70% purity, preferably more than 80%, even more preferably more than 90%, even more preferably more than 95%). The bioactive peptide(s) of the present invention are active against tumor cells, more specifically, against triple negative breast cancer cells. It comprises the SEQ ID 1 (also called herein “3-Nantc”) and one or more of its derivatives (SEQ ID 2, SEQ ID 3, SEQ ID 4, SEQ ID 5, SEQ ID 6, SEQ ID 7, SEQ ID 8, SEQ ID 9, SEQ ID 10). In a more preferable manner, the bioactive peptide has at least more than 70%, or more than 80%, or more than 90% or 100% of identity with SEQ ID 1, SEQ ID 2, SEQ ID 3, SEQ ID 4, SEQ ID 5, SEQ ID 6, SEQ ID 7, SEQ ID 8, SEQ ID 9 or SEQ ID 10.

The term “modulation of tumoral cells” comprises the modulation (increasing, decreasing or maintenance) of at least one of the following parameters in tumoral cells when using the bioactive peptide(s): cellular proliferation, apoptosis, necrosis and the cycle progression when using the bioactive peptide(s). The modulation of tumoral cells is also dependent on the modulation of benign cells, once it is expected that the bioactive peptide(s) are, simultaneously, active against the growth or survival of tumoral cells while having no or few effects over benign cells. In a preferable manner, the modulation of tumoral cells using bioactive peptide(s) decreases the viability of cancer cells while not decreasing the viability of benign skin cells. Moreover, the bioactive peptide(s) decreases the proliferation rate of tumoral cells while not decreasing the proliferation of the benign cells. The bioactive peptide(s) preferably causes a marked difficulty of tumoral cells to proceed to G0/G1 stage, while leads to an arrest in both S and G2/M phases. Moreover, the bioactive peptide(s) do not induce tumor cells death through autophagy. The bioactive peptide(s) induces apoptosis in tumor cells. Also, the bioactive peptide(s) induce very low rates of undesirable necrosis when compared to the induction of necrosis using known drugs for the treatment of tumoral cells, such as Doxorubicin.

The term “therapeutically effective” comprises the use of the bioactive peptide(s) as a sole therapy or in combination with or in addition to other therapies for the modulation of one or more aspects of tumoral cells, while preserving the benign cells in a cancer patient, preferably solid tumor cancer patient, more preferably breast cancer patient, even more preferably triple-negative breast cancer patients. Also, the bioactive peptide(s) may be administered in vivo or in vitro. The bioactive peptide(s) of the present invention is effectively capable of being used in combination with a pharmaceutical composition, including at least one pharmaceutically acceptable vehicle as a carrier, a diluent and/or an excipient.

The protein synthesis is performed through amino acids coupling and high efficiency synthesis in solid phase (HE-SPPS) assisted by microwaves using the equipment Liberty Blue HT12 (CEM Corporation, USA).

The purification of the peptide was performed using HPLC (High efficiency liquid chromatography). Gradients of two eluents are used: deionized water acidified with trifluoroacetic acid - TFA (0.1%) and UV/HPLC grade acetonitrile in TFA (ACN/0.08% TFA, phase B). For detection, an UV detector having an λmáx of 220 nm (215-220 nm absorption range of amide group) is used. The purification is conducted in a semi-preparatory scale (5 mg of brut sample and flux of 1 mL/min) at room temperature. For HPLC, a Varian model Pro Star 210 chromatograph was used with a detector in the ultraviolet region, model Pro Star 330. Columns used: 4.6×250 mm, Boston Green ODS-AQ and loops of 5 µL, 250 µL, respectfully. The results were analyzed by mass spectroscopy (Matrix-Assisted Laser Desorption/lonization) in the ESI-Q-ToF Micromass Micro mass spectroscopy equipment (Micromass, Manchester, UK) and MALDI-ToF/ToFMS (Applied Biosystems, USA). The data obtained was analyzed by the program MassLynx 4.0.

The present invention is illustrated below by reference to the following examples. However, one skilled in the art will appreciate that specific methods and results discussed are merely illustrative of the invention, as innumerable variations, modifications, applications, and extensions of these embodiments and principles can be made without departing from the spirit and scope of the invention.

### EXAMPLES

### Characterization of Peptide (3-NantC) Using MS/MS and HPLC-UV

In order to obtain the peptide purity, an HPLC was performed in these conditions:
Column: 4.6×250 mm, Boston Green ODS-AQ
Solvent A: 0.1% trifluoroacetic in 100% water
Solvent B: 0.1% trifluoroacetic in 100% acetonitrile

| | | |
|---|---|---|
| Gradient: | A | B |
| 0.01 min | 88% | 12% |
| 25 min | 63% | 37% |
| 25.1 min | 0% | 100% |
| 30 min | STOP | |

Flow rate: 1.0 mL/min
Wavelength: 220 nm
Volume: 10 µL

For the confirmation of the sequence, a MS/MS followed the HPLC in these conditions:

| | |
|---|---|
| Probe: ESI | Probe bias: 4.5 kV |
| Nebulizer Gas Floe: 1.5 L/min | |
| Detector: 1.5 kV | |
| CDL:-20.0V | CDL Temp: 250° C. |
| Block Temp:200° C. | |
| T. Flow: 0.2 mL/min | |

B. conc: 50%H2O/50%ACN

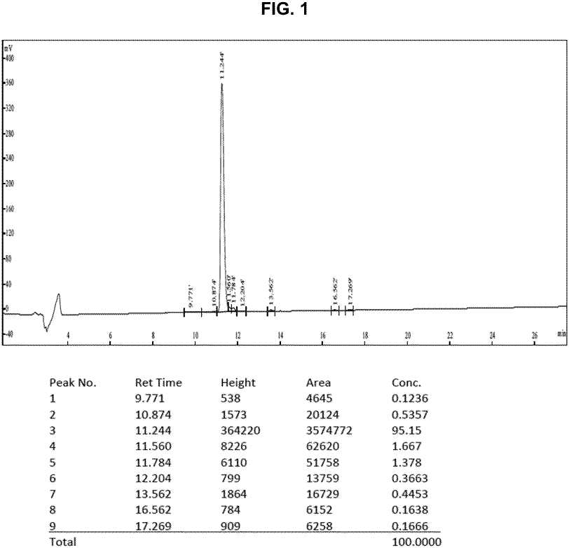

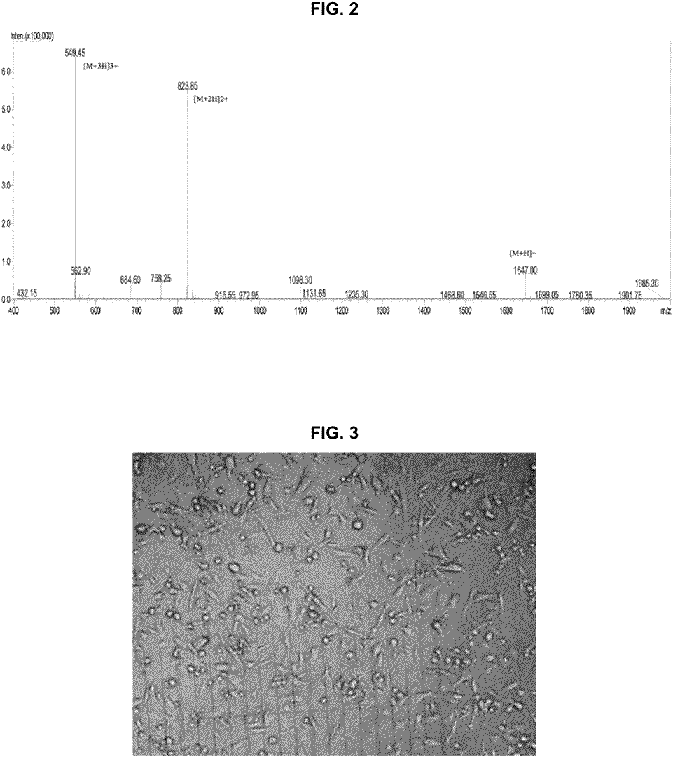

HPLC (FIG. 1) and MS/MS (FIG. 2) characterization of the 3-NAntC peptide (SEQ ID 1) showing purity of 95.15% and molecular mass of 1645.95, confirming the sequence of [H]-MFYPDSRCRGPSET-[OH](SEQ ID 1), also called herein 3-NAntc.

### In Vitro Assays Methods and Results

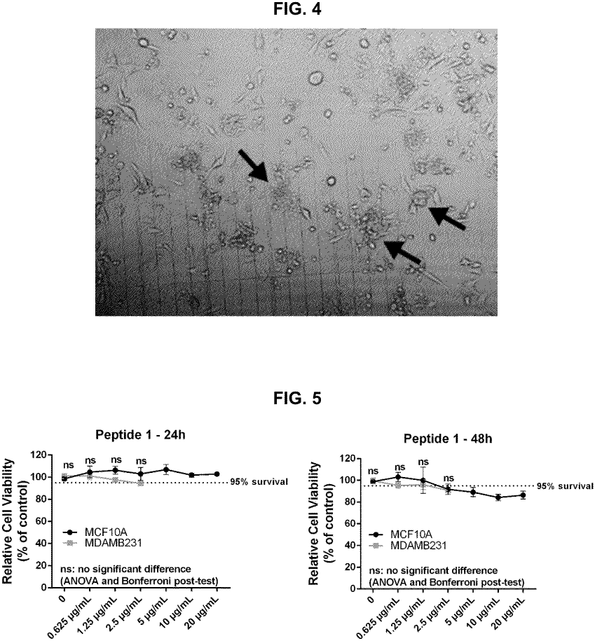

Triple negative breast tumor cells (MDAMB231) are present in lower numbers and have a greater number of vesicles when treated with 3-NAntC for 24 h, as seen in FIG. 4, compared to no treatment (FIG. 3).

### Cell Viability

For the method of MTT, the cells were plated in 96-well sterile plates for cell culture with a cap at the density of 2 × 10⁴ cells/well for treatments lasting 24 h and 1 × 10⁴ cells/well for treatments lasting 48 h or more, completing the volume to 100 µL with culture medium. The plates were incubated for 8-16 h in an incubator at 37° C. and 5% CO₂. The next day, the treatments were prepared in culture medium, applying 200 µL/well at the desired concentration, and the plates were placed in an incubator at 37° C. and 5% CO₂for the duration of treatment. After the treatment period, 20 µL/well of a MTT solution was added in PBS (1×) at a concentration of 5 mg/mL, and the dye was incubated at 37° C. and 5% CO₂ for 2.5 h. After incubation with MTT, Formazan salts were solubilized with the addition of 200 µL of dimethylsulfoxide solution (DMSO): isopropanol (3:1) and agitated for at least 15 minutes at room temperature. After complete dissolution of the salts, the absorbance was read in a spectrophotometer at 570 nm with endpoint protocol.

The calculation of cell viability was performed using the formula:

$$\begin{matrix}\% \text{of cell viability} = \left(\text{O} \text{.D} \text{. cell + test substance}\right) \text{-} \left(\text{O} \text{.D Basal}\right) \times \\ \text{100} \left(\text{O} \text{.D} \text{. cell + culture medium}\right) \text{- O} \text{.D} \text{. Basal}\end{matrix}$$

O.D.: optical density captured by spectrophotometer

NOTE: The Basal O.D. is the one found in the blank wells.

In order to determine if it is the Crotoxin B region that shows a promising antitumor activity (comparison of doses and times for the MDAMB231 cell line) and safety (comparison between the effects on MDAMB231 and MCF10A), the Analysis of Variance (ANOVA) with Bonferroni post-hoc test was applied to the significance of 5% in GraphPad Prism software version 6.0.

MTT shows that peptides [H]-HLLQ FNKMIKFETRKNAIPF-[OH] (Peptide A) and [H)-AIPFYAFY-(OH](Peptide B), both derived from the N-terminal region of Crotoxin B, did not show a promising antitumor activity since they were not able to decrease the viability of the tumor cells or maintain the viability of the benign cells. The first peptide (A) did not decrease the viability of the tumor cells and the second one (B), although it did decrease the MDAMB231 viability at 24 h (viable cells were approximately 82%), it also decreased MCF10A benign cells viability at 48 h (viable cells were approximately 85%) (FIG. 5).

In order to determine the efficacy (comparison of doses and times for the MDAMB231 cell line) and safety (comparison between the effects on MDAMB231 and HDFa cells) of the peptide SEQ ID 1 - MFYPDSRCRGPSET (C-terminal region of Crotoxin B), named 3-NAntC, the Analysis of Variance (ANOVA) with Bonferroni post-hoc test was applied to the significance of 5% in GraphPad Prism software version 6.0.

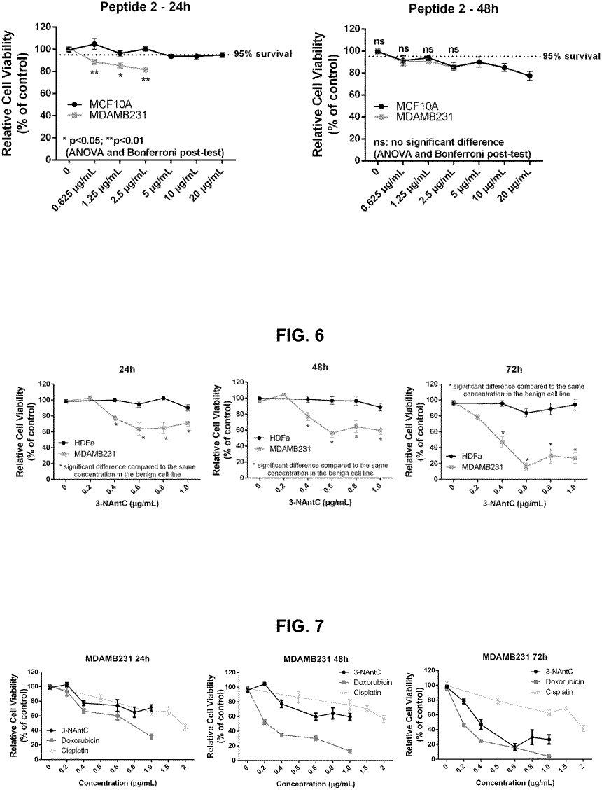

MTT shows that the 3-NAntC significantly decreases the viability of the triple negative cancer cell line (MDAMB231) while not decreasing the viability of benign skin cells — fibroblasts (HDFa) for 24, 48 and 72 h (FIG. 6)-(decrease of tumor viable cells to 70.98%, 59.68% and 17.60%, respectively). The cytotoxicity ratio is on average six tumor cells to one benign (6:1). Data are shown as mean ± SEM of at least three independent assays in triplicate.

MTT also shows that 3-NAntC exceeds the antitumor effect of Cisplatin as early as 48 h of treatment, and the biological effect at 72 h is similar to the effect noted in Doxorubicin at 48 h (FIG. 7). Data are shown as mean ± SEM of at least three independent assays in triplicate.

### Cellular Proliferation

The method of Bomodeoxyuridine (BrdU) quantification was carried out according to the Standard Operational Protocols Nos. 10 and 12 of the Laboratory of Clinical Cytology of the Faculty of Pharmaceutical Sciences of Ribeirao Preto (FCFRP -USP). In this method, for the BrdU quantification, cells were plated in 96-well sterile plates for cell culture with a cap at the density of 2 × 104 cells/well for treatments lasting 24 h and 1 × 104 cells/well for treatments with 48 h or more, completing the volume to 100 µL with culture medium. The plates were incubated for 8-16 h in an incubator at 37° C. and 5% CO₂. The next day, the treatments were prepared in culture medium, applying 200 µL/well at the desired concentration, and the plates were placed in an incubator at 37° C. and 5% CO₂ for the duration of treatment. After the treatment period, 20 µL/well of a BrdU-labeling was added and incubated at 37° C. and 5% CO₂ for 2.5 h. After incubation with BrdU labeling, the plates were stored at 2-4° C. up to 1 week. After storage, the reaction was revealed through ELISA method with an antibody anti-BrdU-POD, followed by washing and substrate solutions. Substrate was developed for 15 minutes and reaction was stopped with HCl 6 M solution and read in a spectrophotometer at 495 nm with endpoint.

The calculation of cell proliferation was performed using the formula:

$$\begin{matrix}\text{\% of cell proliferation} = \left(\text{O} \text{.D} \text{. cell + test substance}\right) \text{-} \left(\text{O} \text{.D Basal}\right) \times \\ \text{100} \left(\text{O} \text{.D} \text{. cell + culture medium}\right) \text{- O} \text{.D} \text{. Basal}\end{matrix}$$

O.D.: optical density captured by spectrophotometer NOTE: The Basal O.D. is the one found in the blank wells.

In order to determine the effects of the peptide in proliferation (MDAMB231 and HDFa cell lines), the Analysis of Variance (ANOVA) with Dunnet and Bonferroni post-hoc tests were applied to the significance of 5% in GraphPad Prism software version 6.0.

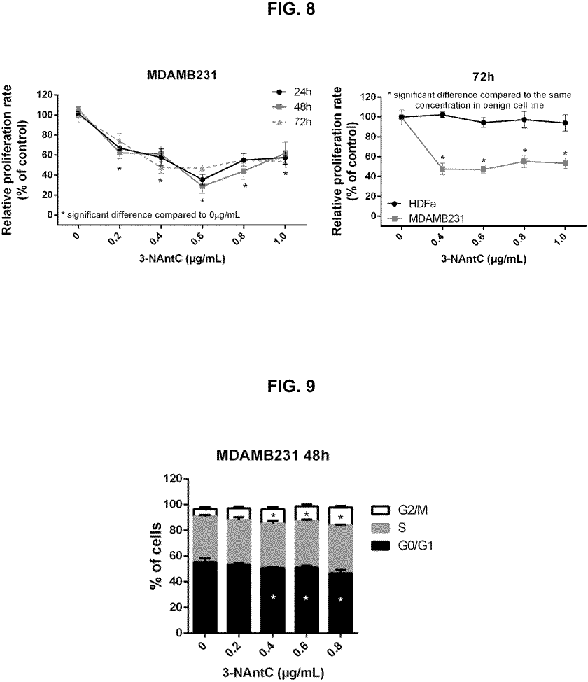

Thus, BrdU labeling showed that 3-NAntC decreased the proliferation rate of MDAMB231 cells (minimum decrease of 33% at 24 h for the 0.2 µg/mL concentration). Moreover, data showed that the peptide did not alter the proliferation of the HDFa benign cell line at the maximum time of exposure (FIG. 8).

### Effects on Cell Cycle Progression of Tumor Cells

For cell cycle progression method, cells were plated in 6-well sterile plates for cell culture with a cap at the density of 2.5 × 10⁵ cells/well for treatments lasting 24 h or more, completing the volume with 1000 µL culture medium. The plates were incubated for 8-16 h in an incubator at 37° C. and 5% CO₂. The next day, the treatments were prepared in culture medium, applying 2000 µL/well at the desired concentration, and the plates were placed in an incubator at 37° C. and 5% CO₂ for the duration of treatment. After the treatment period, cells were collected in a conic 15 mL tube and washed with 1000 µL of PBS (1×). Cells were resuspended in 500 µL/tube of PBS (1×) and 4.5 mL of Ethanol 70°. Then, tubes were closed and kept at 2-4° C. for up to 2 months. Cells were washed with PBS (1×) and resuspended with 135 µL of PBS (1×). A solution of RnaseA at 100 µg/mL was added and incubated at RT for 30 minutes. After incubation, cells were labeled with 2 µL of Propidium Iodide at 100 µg/mL and read at the flow cytometer for size and DNA content.

In order to determine the effects of the peptide in cell cycle progression (MDAMB231 cell line), the Analysis of Variance (ANOVA) with Dunnet post-hoc test was applied to the significance of 5% in GraphPad Prism software version 6.0.

At 48 h, 3-NAntC caused a decrease in cells in G0/G1 (decrease of 8.72% at highest dosage) while increasing G2/M percentage (increase of 8.08% at highest dosage). Thus, this peptide causes a marked difficulty for cells to proceed to G0/G1 stage, while it leads to an arrest of the G2/M phase (see FIG. 9). Data are shown as mean ± SEM of at least three independent assays.

### Cellular Death (Apoptosis/ Necrosis, Necroptosis, Pyroptosis/ Autophagy)

The lactate dehydrogenase (LDH) is a cytosolic enzyme present in most mammalian cells. When there is damage to the plasma membrane, LDH is released into the extracellular matrix and can be quantified, being an important indicator of cell death by necrosis, necroptosis and pyroptosis since LDH extravasation does not occur, for example, in cell death by apoptosis, due to formation of apoptotic bodies and engulfment of material by leukocytes.

For LDH release protocol, after treatments were performed for the MTT protocol, 20 µL of the supernatant of each well was transferred to an EIA/RIA plate. Then, 20 µL/well of the Reaction Mixture from the CyQUANT™ LDH Cytotoxicity Assay kit and reaction was incubated at RT for 30 minutes in the dark. Then, 60 µL/well of the Stop Solution from the kit was added and plate was read in a spectrophotometer at both 490 nm and 650 nm.

The calculation of LDH release was performed as follows:
1. The absorbance measured at 650 nm was subtracted from the absorbance measured at 490 nm.
2. This formula was applied:

$$\begin{matrix}\text{\% of LDH release} = \left(\text{O} \text{.D} \text{. cell + test substance}\right) \text{-} \left(\text{O} \text{.D Basal}\right) \times \\ \text{100} \left(\text{O} \text{.D} \text{. cell + Triton}\right) \text{- O} \text{.D} \text{. Basal}\end{matrix}$$

O.D.: optical density captured by spectrophotometer NOTE: The Basal O.D. is the one found in the blank wells (only reaction mixture and stop solution).

The 3-Methyladenine (3-MA) is an autophagy inhibitor (blocks the activation of this mechanism of action). For molecules that trigger autophagy as a cell death mechanism when this pathway is blocked the molecule loses action.

The MTT method was carried out as previously described herein, comparing the effect of 3-NAntC with or without 3-Methyladenine (3-MA).

Annexin V is a potent marker for apoptosis since it binds to the phosphatidylserine of the cell membrane. This binding only occurs when the phosphatidylserine is externalized in the membrane, which is one of the first signals of apoptosis death. Meanwhile propidium iodide only enters the cells after a membrane rupture and can indicate a final step to cell death. The double labeling with these markers allows to distinguish apoptosis and necrosis death through the following characterization:
Annexin V⁻ / Pl⁻: live cells
Annexin V⁺ / Pl⁻: cells in early apoptosis
Annexin V⁺ / Pl⁺: cell in late apoptosis
Annexin V⁻ / Pl⁺: cells in necrosis

For Apoptosis/ Necrosis protocol, cells were plated 6-well sterile plates for cell culture with a cap at the density of 2.5 × 10⁵ cells/well for treatments lasting 24 h or more, completing the volume with 1000 µL culture medium. The plates were incubated for 8-16 h in an incubator at 37° C. and 5% CO₂. The next day, the treatments were prepared in culture medium, applying 2000 µL/well at the desired concentration, and the plate was placed in an incubator at 37° C. and 5% CO₂ for the duration of treatment. After the treatment period, cells were collected in a conic 15 mL tube and washed with 200 µL of binding buffer (1×) from the FITC Annexin V Apoptosis Detection Kit I. Cells were resuspended in 100 µL/tube of binding buffer (1×) and 1 µL of Annexin V-FITC was added to each tube except the blank. Cells were then incubated for 20 min. in ice and in the dark. Then, 50 µL/tube of binding buffer was added and cells were labeled with 2 µL of Propidium Iodide (PI) at 100 µg/mL and read at the flow cytometer for size and fluorescent content (FITC and PI).

In order to determine the effects of the peptide in LDH release, autophagy induction and apoptosis/necrosis profile of MDAMB231 cell line, the Analysis of Variance (ANOVA) with Dunnet and Bonferroni post-hoc tests were applied to the significance of 5% in GraphPad Prism software version 6.0.

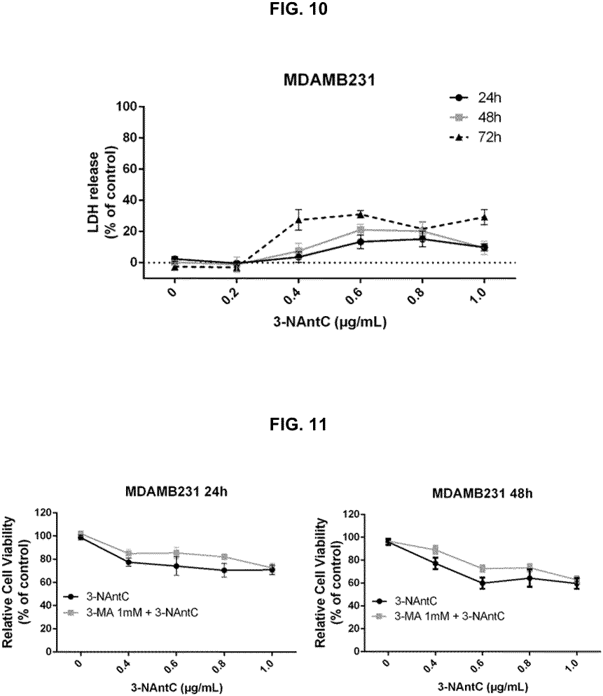

The 3-NAntC does not show any significant increase in LDH release of MDAMB231 cells (FIG. 10), showing no obvious relation to cell death by necrosis, necroptosis and pyroptosis. Data are shown as mean ± SEM of at least three independent assays in triplicate.

The activity of the 3-NAntC with and without the autophagy inhibitor remains the same. Therefore, 3-NAntC does not induce death through autophagy (FIG. 11). Data are shown as mean ± SEM of at least three independent assays in triplicate. This highlights a great difference between the 3-NAntC and Crotoxin B, since the in natura molecule is known to promote autophagy (Yan et. al. Autophagy is involved in cytotoxic effects of crotoxin in human breast cancer cell line MCF-7 cells. Acta Pharmacol Sin, 2007).

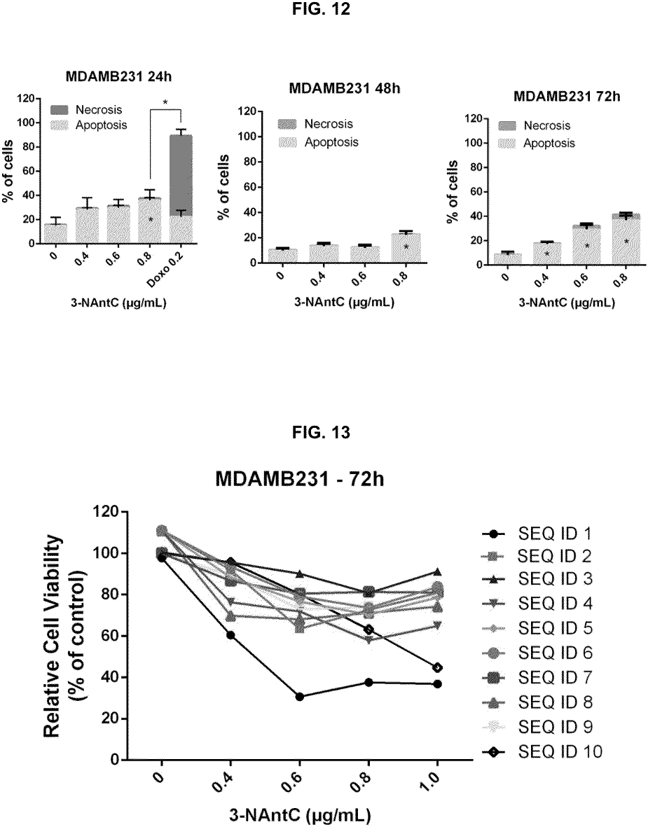

3-NAntC primarily induces apoptosis in tumor cells and very little necrosis compared to Doxorubicin. At 24 and 48 h, 3-NAntC at 0.8 µg/mL increases significantly the apoptosis rate (about 20% in each case), while maintaining the percentage of cells in necrosis. At 72 h 3-NantC increases the apoptosis rate at all tested doses (increase of 28.73% at the highest dosage) (FIG. 12). Data are shown as mean ± SEM of at least three independent assays.

### Derived Peptides - In Vitro Compared Efficacy

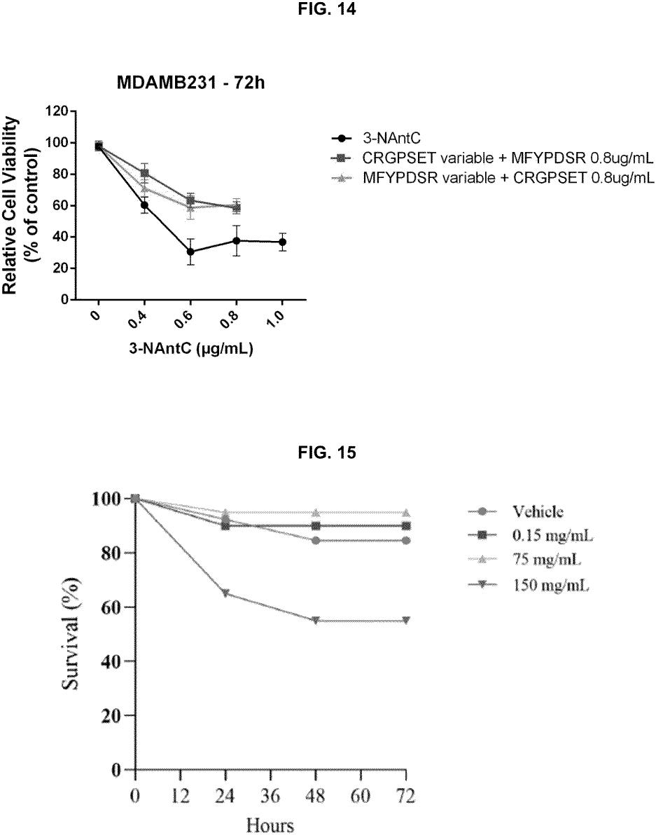

SEQ IDs 2-10 were compared to SEQ ID 1 by MTT assays (as described previously in the in vitro MTT method) in order to determine the best efficacy in decreasing MDAMB231 cells’ viability. Thus, results showed that SEQ ID 1 promotes the highest antitumor effect, followed by SEQ 4 and 8 (FIG. 13). It was observed that SEQ IDs 4 and 5 combined also have a promising antitumor effect (FIG. 14). Moreover, SEQ ID 10 showed that 3-NAntC monomer also has an interesting antitumor effect, with highest concentrations becoming similar to the 3-NAntC effect.

### In Vivo Methods and Results

Transgenic Tg(fli1:EGFP)y1 zebrafish embryos were raised at 28° C. for 48 hours in E3 embryo medium 0.2 mM 1-Phenyl-2-Thiourea aka PTU. Unfertilized eggs or larvae that did not appear healthy or exhibited any obvious developmental defects were excluded before treatment onset. MDA-MB-231 cells were labeled with Dil red fluorescent dye. Approximately 700 Dil Labeled cancer cells were subcutaneously co-implanted into the perivitelline space of 2 days old larvae ± 3-NantC peptide at 75, 100 or 125 mg/mL. Larvae in which tumor cells had been inadvertently injected into circulation or larvae with erroneous implantation of the tumor in the yolk rather than the perivitelline space were excluded from the study. Selected tumor bearing embryos were sorted into experimental groups (20 embryos/group), and pictures of primary tumors were taken right after injection. Doxorubicin treatment was added to embryo water at 2.5 µg/mL according to the study plan. Tumor-bearing embryos were incubated in E3/PTU medium for 72 h at 36° C. After incubation, pictures of the primary tumors and the CVP were taken using a red fluorescent filter. Larvae that died or were lost by other means during the study were excluded from the final analysis.

Images obtained right after implantation (day 0) and after 72 h incubation (day 3) were analyzed by using in-house developed software. Tumor growth regression was calculated and normalized to the negative control group. The number of metastasized cells was counted manually. One-way ANOVA was performed followed by two-tailed Student’s t-test. Definitive outliers were detected using the ROUT method with a Q=1%. Graphs and example images, and experimental data were obtained/analyzed by using GraphPad Prism v9.0.2.

The 3-NAntC peptide is well tolerated by the zebrafish embryos, showing a high embryo survival (see FIG. 15) even at a dose 50,000 times higher than the IC₅₀ for in vitro MDAMB231 experiments at 24 h. Data are shown as mean ± SEM (20 embryos/group). As such, and given the already extremely high dose level, it was not possible to determine the LD₅₀ (dose that would kill 50% of the in vivo model).

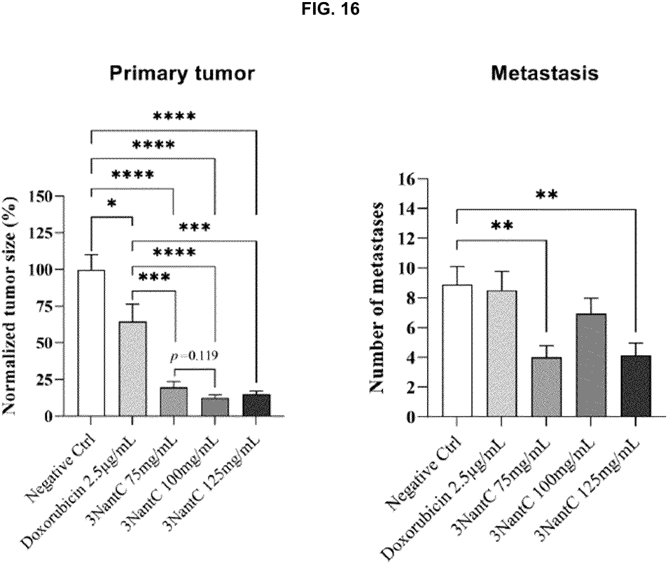

The 3-NantC peptide has an anti-cancer effect on MDAMB231 breast cancer cells at all tested concentrations in vivo; this effect is significantly more potent than the Doxorubicin treatment alone (decrease of 75% of tumor cells against about 35% of Doxorubicin) (see FIG. 16).

The 3-NantC peptide exhibits potent anti-metastatic activity in vivo at 75 and 125 mg/mL on otherwise highly metastatic MDAMB231 breast cancer cells (decrease of about 60% of metastatic cells) (see FIG. 16).

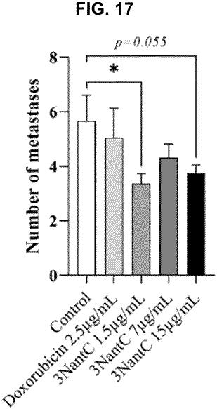

This anti-metastatic effect is observed even in very low concentrations of 3-NAntC (decrease of about 40% in metastatic cells) (see FIG. 17). Data are shown as mean ± SEM (20 embryos/ group) normalized by the control group.

### Mechanism of Action

Western blot protein expression revealed that the 3-NantC peptide may inhibit the activation of pro-survival signals, such as NFKB, while leading to a greater activation of Caspases, namely Caspase 9, when compared to the negative control.

Moreover, proteomics analysis showed that the 3-NantC peptide diminished Rho-GTPases expression (e.g., Rho-A and Rho-C) in addition to continuously disabling the protein elongation mechanism, which strongly indicates the activation of apoptosis (data not shown).

### SEQ ID LIST

SEQ ID 1

MFYPDSRCRGPSET

SEQ ID 2

CMFYPDSRCRGPSETC

(linear)

SEQ ID 3

CMFYPDSRCRGPSETC

(cyclic: disulfide bridge on first and last C; blocked C in the middle)

SEQ ID 4

MFYPDSR

SEQ ID

CRGPSET

SEQ ID 6

CMFYPDSRC

(linear)

SEQ ID 7

CMFYPDSRC

(cyclic: disulfide bridge on first and last C)

SEQ ID 8

CRGPSETC

(linear)

SEQ ID 9

CCRGPSETC

(cyclic: disulfide bridge on first and last C)

SEQ ID 10

MFYPDSRSRGPSET

What is claimed is:

1. A bioactive synthetic peptide derived from the C-terminal portion of the Crotoxin B snake venom toxin having anti-tumoral activity against aggressive solid tumors and little to no toxic effect over benign cells.

2. The bioactive synthetic peptide according to claim 1, wherein the solid tumor is selected from the group consisting of lung, prostate, eye cancer (ocular melanoma), colorectal, skin, brain, pancreas, kidney, breast or any combinations thereof.

3. The bioactive synthetic peptide according to claim 1, wherein the solid tumor is triple negative breast cancer.

4. The bioactive synthetic peptide according to claim 1, wherein the anti-tumoral activity is selected from the group consisting of the modulation of cellular proliferation, decreasing metastasis, apoptosis, necrosis, the cycle of progression and any combinations thereof.

5. The bioactive synthetic peptide according to claim 1, wherein the anti-tumoral activity comprises causing the solid tumor cells difficulty to proceed to GO/G1 stage, while at the same time leading to an arrest of the G2/M phase.

6. The bioactive synthetic peptide according to claim 1, wherein the bioactive peptide is selected from the group consisting of SEQ ID 1, SEQ ID 2, SEQ ID 3, SEQ ID 4, SEQ ID 5, SEQ ID 6, SEQ ID 7, SEQ ID 8, SEQ ID 9, SEQ ID 10 and any combinations thereof.

7. The bioactive synthetic peptide according to claim 6, wherein the bioactive peptide has a purity of between 70% to greater than 95%.

8. The bioactive peptide according to claim 1, wherein the bioactive protein comprises at least 70% similarity with a bioactive peptide selected from the group consisting of SEQ ID 1, SEQ ID 2, SEQ ID 3, SEQ ID 4, SEQ ID 5, SEQ ID 6, SEQ ID 7, SEQ ID 8, SEQ ID 9, SEQ ID 10 and any combinations thereof.

9. The bioactive peptide according to claim 1, wherein the bioactive protein comprises SEQ ID 1.

10. The bioactive peptide according to claim 9, wherein the bioactive protein anti-tumoral activity does not include the promotion of autophagy.

11. A pharmaceutical composition comprising the bioactive peptide according to claim 1 and at least one pharmaceutically acceptable vehicle as a carrier, a diluent and/or an excipient.

12. The pharmaceutical composition according to claim 11 administered in vivo or in vitro.

13. A method of making a bioactive peptide selected from the group consisting of SEQ ID 1, SEQ ID 2, SEQ ID 3, SEQ ID 4, SEQ ID 5, SEQ ID 6, SEQ ID 7, SEQ ID 8, SEQ ID 9, SEQ ID 10 or any combinations thereof, the method comprising the following steps:
a) coupling amino acids using high efficiency synthesis in solid phase assisted by microwaves to obtain the bioactive peptide;
b) purifying the obtained bioactive peptide using HPLC (High efficiency liquid chromatography); and
c) analyzing of the obtained bioactive peptide by mass spectroscopy to confirm the composition of the obtained bioactive peptide.

14. The method according to claim 13, wherein in the purifying step gradients of two eluents are used: (a) deionized water acidified with trifluoroacetic acid - TFA and UV/HPLC grade acetonitrile in TFA.

15. A method of treating a solid tumor is selected from the group consisting of lung, prostate, eye cancer (ocular melanoma), colorectal, skin, brain, pancreas, kidney, breast or any combinations thereof, the method comprising administering a therapeutically effective amount of a bioactive peptide selected from the group consisting of SEQ ID 1, SEQ ID 2, SEQ ID 3, SEQ ID 4, SEQ ID 5, SEQ ID 6, SEQ ID 7, SEQ ID 8, SEQ ID 9, SEQ ID 10 or any combinations thereof in a pharmaceutically acceptable carrier to a patient.

16. The method of treating a solid tumor according to claim 15, wherein the bioactive peptide consists of SEQ ID 1.


SEQUENCE LISTING (10 sequences)

SEQ ID NO: 1 - protein, 14 aa, synthetic construct
MFYPDSRCRGPSET

SEQ ID NO: 2 - protein, 16 aa, synthetic construct
CMFYPDSRCRGPSETC

SEQ ID NO: 3 - protein, 16 aa, synthetic construct
  DISULFID 1..16: note = Disulfide bond
  REGION 1..16: note = Peptide is Circular
CMFYPDSRCRGPSETC

SEQ ID NO: 4 - protein, 7 aa, synthetic construct
MFYPDSR

SEQ ID NO: 5 - protein, 7 aa, synthetic construct
CRGPSET

SEQ ID NO: 6 - protein, 9 aa, synthetic construct
CMFYPDSRC

SEQ ID NO: 7 - protein, 9 aa, synthetic construct
  DISULFID 1..9: note = Disulfide bond
  REGION 1..9: note = Peptide is Circular
CMFYPDSRC

SEQ ID NO: 8 - protein, 8 aa, synthetic construct
CRGPSETC

SEQ ID NO: 9 - protein, 9 aa, synthetic construct
  DISULFID 1..9: note = Disulfide bond
  REGION 1..9: note = Peptide is Circular
CCRGPSETC

SEQ ID NO: 10 - protein, 14 aa, synthetic construct
MFYPDSRSRGPSET